(19) United States
(12) Patent Application Publication
(10) Pub. No.: US 2005/0203168 A1
(43) Pub. Date: Sep. 15, 2005

(54) Angiotensin converting enzyme inhibitor use for treatment and prevention of gastrointestinal disorders

(75) Inventors: Daniel H. Teitelbaum, Ann Arbor, MI (US); Barbara E. Wildhaber, Geneva (CH)

Correspondence Address: David A. Casimir;MEDLEN & CARROLL, LLP, Suite 350, 101 Howard Street, San Francisco, CA, 94105, US

(73) Assignee: The Regents of the University of Michigan, Ann Arbor, MI (US)

(21) Appl. No.: 10/798,470

(22) Filed: Mar. 11, 2004

(51) Int. Cl. A61K 31/401

(52) U.S. Cl. 514/423

(57) ABSTRACT

The present invention provides compositions and methods for treating and preventing inflammatory bowel disease and other gastrointestinal disorders (e.g., short bowel syndrome). In particular, the present invention provides methods of treating symptoms of inflammatory bowel disease and other gastrointestinal disorders using angiotensin converting enzyme (ACE) inhibitors.

This invention was funded, in part, under National Institute of Health Grant AI44076-01. The government may have certain rights in the invention.

### FIELD OF THE INVENTION

The present invention provides compositions and methods for treating and preventing inflammatory bowel disease and other gastrointestinal disorders (e.g., short bowel syndrome). In particular, the present invention provides methods of treating symptoms of inflammatory bowel disease and other gastrointestinal disorders using angiotensin converting enzyme (ACE) inhibitors.

### BACKGROUND OF THE INVENTION

Inflammatory Bowel Disease (IBD) refers to a group of gastrointestinal disorders characterized by a chronic non-specific inflammation of portions of the gastrointestinal tract. Ulcerative colitis and Crohn's Disease are the most prominent examples of IBD in humans. hey are associated with many symptoms and complications, including growth retardation in children, rectal prolapse, blood in stools (e.g., melena and/or hematochezia), wasting, iron deficiency, and anemia (e.g. iron deficiency anemia and anemia of chronic disease or of chronic inflammation).

The etiology (or etiologies) and pathogenesis of IBD are still unclear. Previous understanding of the pathogenesis was limited to a three-stage process: (a) an irritant, which could be an immune process or infectious agent, activates (b) leukocytes which release enzymes such as pro-inflammatory cytokines (particularly tumor necrosis factor alpha (TNA-α), proteases and inflammatory mediators such as histamine, serotonin and prostaglandins, and (c) these products cause edema, pain, heat and loss of function. See Wyngaarden and Smith (eds.) Cecil's Textbook of Medicine (W. B. Saunders Co. 1985), Berkow (ed.). The Merck Manual of Diagnosis and Therapy (Merck Sharp & Dohme Research Laboratories, 1982), and Harrison's Principles of Internal Medicine, 12th Ed., McGraw-Hill, Inc. (1991).

Numerous theories implicate multiple factors leading up to IBD including genetic predisposition, environmental factors, infectious agents and immunologic alterations. See Kirsner, J. B., et al. (eds), Inflammatory Bowel Disease, 3rd ed., Lea and Febiger, Philadelphia (1988); Zipser, R. D., (ed.), Dig. Dis. Sci., 33 Suppl.:1S-87S (1988). The immunologic alterations in IBD appear to be autoimmune in nature, with colonic autoantibodies and lymphocyte-cytotoxicity directed against colonic epithelial cells. However, even the latest developments in the immunologic aspects of the pathogenesis of IBD cannot answer the basic question, i.e., whether the detected changes in humoral and cellular immunity reflect a primary defect or secondary response to injury.

Treatment for IBD currently includes steroids, sulphasalazine and its derivatives, and immunosuppressive drugs such as cyclosporin A, mercaptopurine and azathioprine. Such therapies are directed toward suppression of the general immune response. These approaches often result in poor success, have little or no selectivity, and can be accompanied by unwanted and sometimes dangerous consequential side effects.

Thus, there exists a need for effective treatment, both prophylactic and curative, for IBD and related conditions. Such a therapy should be specific and should not be accompanied by unwanted side effects.

### SUMMARY OF THE INVENTION

The present invention provides methods for treating and preventing inflammatory bowel disease and related conditions (e.g., short bowel syndrome). In particular, the present invention provides methods of treating symptoms of inflammatory bowel disease and related conditions using angiotensin converting enzyme (ACE) inhibitors.

Accordingly, the present invention provides a method of treatment, comprising providing a subject having a symptom of inflammatory bowel disease or a related condition and a therapeutic composition comprising ACE inhibitors, and; administering the composition to the subject under conditions such that the manifestations of the condition are reduced. It is not intended that the present invention be limited to any particular subject. Indeed, a variety of subjects are contemplated. In one embodiment, the subject is a mammal. In a preferred embodiment, the subject is a human.

In some embodiments, the composition is administered to the subject orally. In other embodiments, the composition is administered to the subject parenterally, topically, intravenously, or rectally. In still further embodiments, the composition comprises a transdermal patch.

In a preferred embodiment, the subject possesses symptoms of inflammatory bowel disease. In some embodiments, the subject is suffering from Crohn's disease. In other embodiments, the subject is suffering from ulcerative colitis. In still further embodiments, the subject is suffering from irritable bowel syndrome, celiac disease, stomach ulcers, diverticulitis, pouchitis, proctitis, or chronic diarrhea. In a preferred embodiment, the administration of a therapeutic composition comprising ACE inhibitors reduces the symptoms of disease (e.g. reduces the symptoms of inflammatory bowel disease, Crohn's disease, ulcerative colitis, irritable bowel syndrome, celiac disease, stomach ulcers, diverticulitis, pouchitis, proctitis, or chronic diarrhea). In some embodiments, the composition comprising the ACE inhibitors is administered under conditions such that the symptoms of IBD are reduced. In other embodiments, the subject is at risk for inflammatory bowel disease, and the therapeutic composition is administered prophylactically. In still further embodiments, a therapeutically effective amount of ACE inhibitors is administered to the subject. In some embodiments, the ACE inhibitor is administered in conjunction with one or more other therapeutic compounds (e.g., known therapeutic compounds).

It is not intended that the present invention be limited to particular ACE inhibitor. Indeed, a variety of ACE inhibitors are contemplated. In one embodiment, the ACE inhibitor is enalaprilat. In other embodiments, the ACE inhibitor is selected from the group consisting of alacepril, benazepril (Lotensin), captopril (Capoten), cilazapril, ceranapril, delapril, enalapril (Vasotec), fosinopril (Monopril), fosinoprilat, imidapril, lisinopril (Prinivil, Zestril), moexipril (Univasc), perindopril (Aceon), perindoprilat, quinapril (Accupril), quinaprilat, ramipril (Altace), saralasin acetate, spirapril, temocapril, trandolapril (Mavik), Bioproject BP1.137, Chiesi CHF 1514, Fisons FPL-66564, idrapril, Marion Merrell Dow MDL-100240, and Servier S-5590.

The present invention also provides a method of treatment, comprising, providing a subject at risk for inflammatory bowel disease and other non-inflammatory gastrointestinal disorders (e.g., short bowel syndrome) and a composition comprising ACE inhibitors, and administering to the subject a therapeutically effective amount of the composition so as to prevent the subject from experiencing symptoms of inflammatory bowel disease, related inflammatory conditions of the gastrointestinal tracts, or other gastrointestinal disorders. In a preferred embodiment, the administration of the therapeutic composition comprising ACE inhibitors delays the progression of the symptoms of inflammatory bowel disease. In one embodiment, the subject at risk for inflammatory bowel disease is a human. In a preferred embodiment, the human is selected from a young adult, a person living in the United States, a person living in England, a person living in Northern Europe, a person of Jewish descent, a person living in a developing nation, a person with family members who suffer from inflammatory bowel disease or a person determined to carry an inflammatory bowel disease risk gene. In a particularly preferred embodiment, the administration of the therapeutic composition comprising ACE inhibitors prevents the onset of one or more symptoms of inflammatory bowel disease (e.g. prevents the onset of abdominal pain, diarrhea, rectal bleeding, weight loss, fever, loss of appetite, dehydration, anemia, or malnutrition, or any combination thereof).

In some embodiments, the composition is administered orally to the subject at risk for inflammatory bowel disease orally. In other embodiments, the composition is administered to the subject parenterally, topically, intravenously, or rectally. In still further embodiments, the composition comprises a transdermal patch.

In some embodiments, the subject at risk for developing gastrointestinal inflammation or inflammatory bowel disease is at risk for developing Crohn's disease, irritable bowel syndrome, celiac disease, ulcerative colitis, stomach ulcers, diverticulitis, pouchitis, proctitis or chronic diarrhea.

In some embodiments, the compositions of the invention are administered to infants, children, and adults who suffer from short bowel syndrome. In this condition, the subject is lacking a sufficient length of the gastrointestinal tract to permit the normal absorption of fluids, electrolytes and nutrients to permit growth and survival. In such a condition, the subject requires supplementation with either specialized enteral or parenteral nutrients. The use of angiotensin converting enzyme inhibitors, in some embodiments of the present invention, is administered to such individuals to facilitate growth and adaptation of the gastrointestinal tract. In such embodiments, the compositions of the present invention are used to treat or improve the disorder of short bowel syndrome, although an understanding of the mechanism is not necessary to practice the present invention and the invention is not limited to any particular mechanism of action.

In some embodiments, the compositions of the invention are administered orally to a subject at risk for short bowel syndrome orally. In other embodiments, the compositions are administered to the subject parenterally, topically, intravenously, or rectally. In still further embodiments, the composition comprises a transdermal patch.

### DESCRIPTION OF THE DRAWINGS

FIG. 1 depicts intraepithelial lymphocyte ACE protein expression in Sham mice, mice after massive small bowel resection, and mice with massive small bowel resection administered ACE inhibitors.

FIG. 2 depicts epithelial cell apoptosis in Sham mice (defined as mice undergoing a transaction of the mid-portion of the small bowel with no resection of intestine), SBS mice (defined as mice undergoing a massive (70%) small bowel resection, and SBS mice administered ACE inhibitors. For all figures, Enalaprilat was used as the angiotensin converting enzyme inhibitor (ACE-I), at a dose of 0.6 mg/kg/day intraperitoneally. Epithelial cell (EC) apoptosis is shown 1 week postoperatively in each group. In the Sham and SBS group EC apoptosis was significantly increased. With ACE-I administration a significant decrease in apoptosis was noted. Apoptosis was defined by Annexin V staining and detected with flow cytometry, whereby Annexin V positive cells denote apoptosis (circled quadrants), and propidium iodide positive cells denote cell necrosis. Note the marked reduction in apoptosis in the angiotensin converting enzyme inhibitor treated mice. Levels of apoptosis for each group are as follows: Sham: 39.9±5.7%; SBS: 36.0±19.9%; SBS/ACE-I 14.4±5.1%. The difference was significantly different (using ANOVA, with a least significant difference post-hoc testing) at the P<0.01, level.

FIG. 3 depicts the one-week postoperative histology of Sham mice, SBS mice, and SBS mice administered ACE inhibitors. With ACE-I administration crypt depth significantly further increased, whereas villus height was unchanged. Magnification 160×. Mean villus heights in each group are as follows (expressed as μm±standard deviation): Sham: 353±40; SBS: 556±66*; Sham/ACE-I 340±20; SBS/ACE-I 552±56. Mean crypt depths in each group are as follows (expressed as μm±standard deviation): Sham: 77±10; SBS: 113±14*; Sham/ACE-I 87±6; SBS/ACE-I 145±22°. Although not shown in the figure, the Sham/ACE-I group depicts a separate group of Sham mice treated with ACE-I, and denotes that the action of ACE-I is most evident in mice with the disorder of short bowel syndrome (SBS). *P<0.05 Sham vs. SBS, °P<0.05 SBS vs. SBS+ACE-I, using analysis of variance. Note predominant action of ACE is in the increase of crypt depth—the key portion of the intestine for early-renewed intestinal growth.

FIG. 4 depicts intraepithelial lymphocyte TNF-alpha mRNA expression in Sham mice, mice after massive small bowel resection, and mice with massive small bowel resection administered ACE inhibitors. TNF-α (tumor necrosis factor alpha) mRNA is expressed using polymerase chain reaction techniques. Statitical comparison of groups used analysis of variance with P<0.05 between marked groups.

FIG. 5 depicts changes in the histology of a mouse inflammatory bowel disease model. Mice were fed DSS for a 5-day period. This well established model induces a colitis (i.e., inflammation of the colon). In this setting, one group of mice were additionally given the angiotensin converting enzyme—Enalaprilat, the other group (Control group) were given saline as a control. The histomicrographs show a more severe degree of colitis in the Control group. In images 1 and 2 (DSS-saline images 1 and 2) representative images from DSS-induced colitis treated only with saline injection are shown, at 200× and 100× magnification. Note the essentially complete loss of all colonic mucosal crypts and nearly complete loss of surface epithelial cells. This corresponds grossly to areas of ulceration in this model. Images 3 and 4 (ACE-I images 1 and 2) depict samples of mice with DSS colitis treated with daily enalaprilat injection, at 200× and 100× magnification, respectively. Note that while there are areas of ulcerative mucosa, there are also significant areas retaining colonic crypts as well as surface epithelial cells.

FIG. 6 shows that the time course of development of heme-positive stools in mice treated with ACE-I was significantly delayed in comparison to mice treated only with saline injection.

### DEFINITIONS

To facilitate an understanding of the present invention, a number of terms and phrases are defined below:

“Gastrointestinal inflammation” as used herein refers to inflammation of a mucosal layer of the gastrointestinal tract, and encompasses acute and chronic inflammatory conditions. Acute inflammation is generally characterized by a short time of onset and infiltration or influx of neutrophils. Chronic inflammation is generally characterized by a relatively longer period of onset and infiltration or influx of mononuclear cells. Chronic inflammation can also typically characterized by periods of spontaneous remission and spontaneous occurrence. “Mucosal layer of the gastrointestinal tract” is meant to include mucosa of the bowel (including the small intestine and large intestine), rectum, stomach (gastric) lining, oral cavity, and the like.

“Chronic gastrointestinal inflammation” refers to inflammation of the mucosal of the gastrointestinal tract that is characterized by a relatively longer period of onset, is long-lasting (e.g., from several days, weeks, months, or years and up to the life of the subject), and is associated with infiltration or influx of mononuclear cells and can be further associated with periods of spontaneous remission and spontaneous occurrence. Thus, subjects with chronic gastrointestinal inflammation may be expected to require a long period of supervision, observation, or care. “Chronic gastrointestinal inflammatory conditions” (also referred to as “chronic gastrointestinal inflammatory diseases”) having such chronic inflammation include, but are not necessarily limited to, inflammatory bowel disease (IBD), colitis induced by environmental insults (e.g., gastrointestinal inflammation (e.g., colitis) caused by or associated with (e.g., as a side effect) a therapeutic regimen, such as administration of chemotherapy, radiation therapy, and the like), colitis in conditions such as chronic granulomatous disease (Schappi et al., Arch. Dis. Child., 1984:147 (2001)), celiac disease, celiac sprue (a heritable disease in which the intestinal lining is inflamed in response to the ingestion of a protein known as gluten), food allergies, gastritis, infectious gastritis or enterocolitis (e.g., Helicobacter pylori-infected chronic active gastritis) and other forms of gastrointestinal inflammation caused by an infectious agent, and other like conditions.

The acute and chronic inflammation is secondary to an increase in pro-inflammatory cytokines (particularly tumor necrosis factor-alpha) and an increase in epithelial cell apoptosis. The resultant manifestations of these factors are a loss of the mucosal epithelial lining and the above stated neutrophil/monocyte infiltrate.

As used herein, “inflammatory bowel disease” or “IBD” refers to any of a variety of diseases characterized by inflammation of all or part of the intestines. Examples of inflammatory bowel disease include, but are not limited to, Crohn's disease and ulcerative colitis. Reference to IBD throughout the specification is often referred to in the specification as exemplary of gastrointestinal inflammatory conditions, and is not meant to be limiting.

As used herein, the term “symptoms of IBD” is herein defined to detected symptoms such as abdominal pain, diarrhea, rectal bleeding, weight loss, fever, loss of appetite, and other more serious complications, such as dehydration, anemia and malnutrition. A number of such symptoms are subject to quantitative analysis (e.g. weight loss, fever, anemia, etc.). Some symptoms are readily determined from a blood test (e.g. anemia) or a test that detects the presence of blood (e.g. rectal bleeding). The term “wherein said symptoms are reduced” refers to a qualitative or quantitative reduction in detectable symptoms, including but not limited to a detectable impact on the rate of recovery from disease (e.g. rate of weight gain). The diagnosis is typically determined by way of an endoscopic observation of the mucosa, and pathologic examination of endoscopic biopsy specimens.

As used herein, the term “a therapeutically effective amount” of a composition comprising ACE inhibitors is herein defined as the dosage level required for a subject such that the subject's symptoms of IBD are reduced.

As used herein, the phrase “under conditions such that the symptoms are reduced” refers to any degree of qualitative or quantitative reduction in detectable symptoms of IBD, including but not limited to, a detectable impact on the rate of recovery from disease (e.g. rate of weight gain), or the reduction of at least one of the following symptoms: abdominal pain, diarrhea, rectal bleeding, weight loss, fever, loss of appetite, dehydration, anemia, distention, fibrosis, inflamed intestines and malnutrition.

As used herein, the term “at risk for IBD” is herein defined as encompassing the segment of the world population that has an increased risk (i.e. over the average person) for IBD and can occur at any age. It occurs worldwide, but is most common in the United States, England, and northern Europe. It is especially common in people of Jewish descent. An increased frequency of this condition has been recently observed in developing nations. Increased risk is also most prevalent in people with family members who suffer from inflammatory bowel disease.

As used herein, the term “therapeutic composition comprising ACE inhibitors” refers to compositions containing ACE inhibitors together with one or more other compounds or agents including, but not limited to, other ACE inhibitors, other therapeutic agents, physiologically tolerable liquids, gels, carriers, diluents, adjuvants, excipients, salicylates, steroids, immunosuppressants, antibodies, cytokines, antibiotics, binders, fillers, preservatives, stabilizing agents, emulsifiers, and buffers.

As used herein, the term “continuous manner” when used in reference to the method of delivery or administration of the therapeutic composition comprising ACE inhibitors of the present invention is defined as meaning a substantially uninterrupted administration such that a therapeutic dosage is stretched over a period of time and avoids dosage spiking that is common among other modes of administration (e.g. oral administration or intravenous administration). Examples of modes of administration that employ a continuous manner of delivery include, but are not limited to, a transdermal patch, a suppository, or a slow release oral formulation. As used herein, the term “subject” refers to a patient that is administered the therapeutic composition comprising ACE inhibitors of the present invention. Examples of subjects, include, but are not limited to, humans and other mammals such as non-human primates, horses, dogs, and cats.

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

“Short Bowel Syndrome” refers to the condition of the gastrointestinal tract whereby the bowel is lacks an adequate length to allow for the sufficient length to absorb nutrients, fluids and/or electrolytes.

“Intestinal Failure” refers to the condition of a gastrointestinal tract which lacks sufficient ability to absorb nutrients, fluids and electrolytes to sustain an organism, and to provide for growth. The condition includes, but is not limited, to conditions of short bowel syndrome, malabsorption, overwhelming inflammatory conditions of the bowel, and dymotility syndromes (conditions whereby the bowel lacks adequate mobility to propel nutrients through the intestine).

### DETAILED DESCRIPTION OF THE INVENTION/PREFERRED EMBODIMENTS

The present invention provides compositions and methods for treating and preventing inflammatory bowel disease and related conditions. In addition, the present invention provides compositions and methods for the treatment of short bowel syndrome (SBS) and other related problems of intestinal failure. In particular, the present invention provides methods of treating symptoms of inflammatory bowel disease, short bowel syndrome, intestinal failure and related conditions using angiotensin converting enzyme (ACE) inhibitors.

Experiments conducted during the development of the present invention demonstrated that the ACE inhibitors find use in the treatment and prevention of gastrointestinal inflammation diseases and conditions, including inflammatory bowel disease. Additional experiments demonstrate that use of ACE inhibitors may be effective in improving intestinal adaptation in cases of the short bowel syndrome, and may be used as a therapeutic modality in such conditions. Thus, the present invention provides new compositions and methods for using ACE inhibitors in the treatment and prevention of such conditions. The ACE inhibitors of the present invention may be used alone, or in combination with any other known or later identified treatment or intervention for such gastrointestinal inflammation diseases and short bowel syndrome conditions.

Examples of ACE inhibitors that find use in the compositions and methods of the present invention include, but are not limited to, Beecham's BRL 36,378 as disclosed in European patent Nos. 80822 and 60668; Chugai's MC-838 disclosed in CA. 102:72588v and Jap. J. Pharmacol. 40:373 (1986); Ciba-Geigy's CGS 14824 (3-([1-ethoxycarbonyl-3-phenyl-(1S)-propyl]-amino)-2,3,4,5-tetrahydro-2-oxo-1-(3S)-benzazepine-1 acetic acid HCl) disclosed in U.K. Patent No. 2103614 and CGS 16,617 (3(S)-[[(1S)-5-amino-1-carboxypentyl]amino]-2,3,4,5-tetrahydro-2-oxo-1H-1-benzazepine-1-ethanoic acid) disclosed in U.S. Pat. No. 4,473,575; cetapril (alacepril, Dainippon) disclosed in Eur. Therap. Res. 39:671 (1986); 40:543 (1986); ramipril (Hoechst) disclosed in Eur. Patent No. 79-022 and Curr. Ther. Res. 40:74 (1986); Ru 44570 (Hoechst) disclosed in Arzneimittelforschung 35:1254 (1985), cilazapril (Hoffinan-LaRoche) disclosed in J. Cardiovasc. Pharmacol. 9:39 (1987); R.sub.O 31-2201 (Hoffman-LaRoche) disclosed in FEBS Lett. 165:201 (1984); lisinopril (Merck) disclosed in Curr. Therap. Res. 37:342 (1985) and Eur. patent appl. No. 12-401, indalapril (delapril) disclosed in U.S. Pat. No. 4,385,051; rentiapril (fentiapril, Santen) disclosed in Clin. Exp. Pharmacol. Physiol. 10:131 (1983); indolapril (Schering) disclosed in J. Cardiovasc. Pharmacol. 5:643, 655 (1983); spirapril (Schering) disclosed in Acta. Pharmacol. Toxicol. 59 (Supp. 5):173 (1986); perindopril (Servier) disclosed in Eur. J. Clin. Pharmacol. 31:519 (1987); quinapril (Warner-Lambert) disclosed in U.S. Pat. No. 4,344,949 and CI 925 (Warner-Lambert) ([3S-[2[R(*)R(*)]]3R(*)]-2-[2-[[1-(ethoxy-carbonyl)-3-phenylpropyl]amino[-1-oxopropyl]-1,2,3,4-tetrahydro-6,7-dimethoxy-3-isoquinolinecarboxylic acid HCl) disclosed in Pharmacologist 26:243, 266 (1984), WY-44221 (Wyeth) disclosed in J. Med. Chem. 26:394 (1983), each of which is hereby incorporated by reference in its entirety.

In carrying out the method of the invention, the angiotensin converting enzyme inhibitor alone or in combination with other drugs may be incorporated in a single conventional dosage form or each compound may be incorporated into a separate conventional dosage form to be taken at the same time. The dosage forms may comprise conventional oral forms, rectal forms or parenteral forms, such as tablets, capsules, suppositories, powders, ampoules, elixirs, suspensions, solutions, syrups, sustained release preparations and fluid injectable forms, such as sterile solutions. Oral dosage forms are preferred, although parenteral forms, such as intramuscular, intraperitoneal or intravenous forms are quite satisfactory as well.

The dose administered may be adjusted according to age, weight and condition of the patient, as well as the route of administration, dosage form and regimen and the desired result.

Thus, for oral administration, a satisfactory result may be obtained employing the ACE inhibitor in an amount within the range of from about 0.01 mg/kg to about 100 mg/kg and preferably from about 0.1 mg/kg to about 25 mg/kg, and when employing ceronapril preferably from about 0.01 to about 10 mg/kg, alone or in combination with other drugs in an amount within the range of from about 0.01 mg/kg to about 100 mg/kg and preferably from about 0.1 mg/kg to about 25 mg/kg with the ACE inhibitor and other drugs being employed together in the same oral dosage form or in separate oral dosage forms taken at the same time.

A preferred oral dosage form, such as tablets or capsules, will contain the ACE inhibitor in an amount of from about 1 to about 500 mg, preferably from about 125 to about 200 mg, and more preferably from about 25 to about 150 mg.

For parenteral administration, the ACE inhibitor will be employed in an amount within the range of from about 0.001 mg/kg to about 10 mg/kg and preferably from about 0.01 mg/kg to about 1 mg/kg.

The composition described above may be administered in the dosage forms as described above in single or divided doses of one to multiple times daily, or continuously or semi-continuously (See e.g., U.S. Pat. No. 6,267,990 for controlled release techniques, herein incorporated by reference in its entirety). It may be advisable to start a patient on a low dose combination and work up gradually to a high dose combination.

Tablets of various sizes can be prepared, e.g., of about 50 to 700 mg in total weight, containing one or more active substances in the ranges described above, with the remainder being a physiologically acceptable carrier of other materials according to accepted pharmaceutical practice. These tablets can, of course, be scored to provide for fractional doses. Gelatin capsules can be similarly formulated.

Liquid formulations can also be prepared by dissolving or suspending one or the combination of active substance in a conventional liquid vehicle acceptable for pharmaceutical administration so as to provide the desired dosage.

In formulating the compositions of this invention, the active substances, in the amounts described above, are compounded according to accepted pharmaceutical practice with a physiologically acceptable vehicle, carrier, excipient, binder, preservative, stabilizer, flavor, etc., in the particular type of unit dosage form.

Illustrative of the adjuvants that may be incorporated in tablets are the following: a binder such as gum tragacanth, acacia, corn starch or gelatin; an excipient such as dicalcium phosphate or cellulose; a disintegrating agent such as corn starch, potato starch, alginic acid or the like; a lubricant such as stearic acid or magnesium stearate; a sweetening agent such as sucrose, lactose or saccharin; a flavoring agent such as orange, peppermint, oil of wintergreen or cherry. When the dosage unit form is a capsule, it may contain in addition to materials of the above type a liquid carrier such as a fatty oil. Various other materials may be present as coatings or to otherwise modify the physical form of the dosage unit. For instance, tablets or capsules may be coated with shellac, sugar or both. A syrup of elixir may contain the active compound, water, alcohol or the like as the carrier, glycerol as solubilizer, sucrose as sweetening agent, methyl and propyl parabens as preservatives, a dye and a flavoring such as cherry or orange.

Many of the active substances described above form commonly known, pharmaceutically acceptable salts such as alkali metal and other common basic salts or acid addition salts, etc. References to the base substances are therefore intended to include those common salts known to be substantially equivalent to the parent compound.

### EXPERIMENTAL

The following examples are provided in order to demonstrate and further illustrate certain preferred embodiments and aspects of the present invention and are not to be construed as limiting the scope thereof.

### EXAMPLE 1

### Materials and Methods

Animals: Male, 2 month old, C57BL/6J mice (Jackson Laboratories, Bar Harbor, Me.) were maintained in a 12-hour day-night rhythm at 23° C. and a relative humidity of 40%-60%. 24 hours before surgery chow was exchanged to micro-stabilized rodent liquid diet (TestDiet, Richmond, Ind.). Anesthesia was achieved using sodium pentobarbital (50 mg/kg body weight, intraperitoneally, i.p.). For some experiments B6.129P2-Ace^{tm2Unc} (ACE^{-/-}; lacking somatic ACE, generous gift from the Dr. O. Smithies, Duke University), or B6;129S6-Tnf^{tm1Gkl} (TNF-α-knockout mice, Jackson Labs) were used. Studies conformed, and were approved by the University Committee on Use and Care of Animals at the University of Michigan.

Short Bowel Syndrome Model: A 70% mid-small bowel resection was performed (i.e. bowel between 3.5 cm distal to the ligament of Treitz and 3.5 cm proximal to the ileocecal valve), followed by an end-to-end jejuno-ileal anastomosis with 7-0 monofilament, absorbable suture. Postoperatively a subcutaneous bolus of 3 ml 0.9% saline solution was given to maintain hydration status.

Sham operation: Mid-small bowel was transsected and re-anastomosed without bowel-resection.

Post-Surgery: Postoperatively ad libitum water and liquid diet were reintroduced. If mice were not sacrificed after 10 days, food was changed to standard chow. Four groups were studied (N=6 per group): 1 week (SBR 1 w, Sham 1 w) or 4 weeks (SBR 4 w, Sham 4 w), to assess temporal changes. Animals were sacrificed using C0₂, and the intestine was harvested.

Histology: A 0.5 cm segment of mid-small bowel was fixed in 10% formaldehyde, and processed for hematoxylin and eosin staining. Villus height and crypt depth were measured using a calibrated micrometer. Each measurement consisted of a mean of 16 different low power fields.

Epithelial cell apoptosis assays: Two methods were used to assess EC apoptosis.

Annexin V staining: Apoptosis was determined by flow cytometry based on the cell surface expression of phosphatidylserine using Annexin V staining (BD PharMingen, San Diego, Calif.). Propidium iodide was utilized to simultaneously monitor cell necrosis. Flow cytometry was performed by using standard techniques. Separation of EC from IEL was done by forward- and side-scatter gating characteristics. Further confirmation of EC and IEL purity was performed with specific staining of EC with antibody generated by a hybridoma G8.8 and a pan-lymphoid antibody (anti-CD45, PharMingen, San Diego, Calif.): Purity exceeded >98%.

TUNEL staining: Paraffin-embedded tissue was assayed with TUNEL (Terminal deoxynucleotidyl transferase Biotin-dUTP Nick End Labeling) staining, according to slight modification of manufacturer's instructions (ApopTag InSitu Apoptosis Detection Kit, Serological Corporation, Norcross, Ga.): slides were incubated with only ⅓ of the recommended concentration of TdT enzyme, in order to avoid over-staining. Apoptosis rate (percent of EC showing apoptosis) was assessed with TUNEL staining and by morphological criteria (nuclear margination, chromatin and cytoplasmic condensation, shrinkage from neighboring cells, and formation of apoptotic bodies with nuclear and cytoplasmic fragmentation). Each assessment of apoptosis consists of the mean of 8 different crypt-villus-complexes.

Mucosal cell isolation: Isolation of mucosal cells was performed using standard protocols. This included isolation of cells with an extraction buffer (1 mM EDTA, 1 mM dithiotheritol in phosphate buffer saline), and purification in 20% isotonic Percoll (Pharmacia, Piscataway, N.J.). Viability exceeded 95% using trypan blue exclusion staining. The cell suspension contained a purified mixture of EC and IEL at a ratio of 60:40.

IEL purification: Isolation of purified IEL from EC was performed by direct magnetic separation. Magnetic beads conjugated with antibody to CD45 (pan-lymphoid marker) were used to segregate IEL from EC (BioMag SelectaPure Anti-Mouse CD 45R Antibody Particles, Polyscience Inc., Warrington, Pa.). Magnetic separation was performed twice to further deplete EC. Final IEL purity was greater than 99% by flow cytometry.

IEL staining and sorting: IEL were stained with antibodies to T-cell receptor (TCR)-αβ (H57, Invitrogen Corporation, Gibco BRL, Carlsbad, Calif.), TCR-γδ (GL3, Invitrogen), CD4 (RM4-5, BD PharMingen, San Diego, Calif.) or CD8α (53-6.7, PharMingen). Isotype control antibodies were used to adjust gating. IEL sub-populations were sorted using an EPICS Elite (Coulter, Miami, Fla.) flow cytometer. Sorted IEL were kept at 4° C. until RNA isolation.

Isolation of total RNA: A guanidine isothiocyanate/chloroform extraction method was performed using Trizol (Gibco BRL, Gaithersburg, Md.) according to manufacturer's directions.

Microarray assay: Total IEL-RNA was purified with the RNeasy Mini Kit (Qiagen, Valencia, Calif.). Affymetrix system microarray chips (U74 set, Affyrmetrix, Inc., Santa Clara, Calif.) allowed simultaneous interrogation of 12,491 full-length mouse genes and EST clusters from the UniGene database. Hybridization and analysis were performed by the University of Michigan NIDDK Biotechnology Center. Probe-pair measures were obtained from the images of each chip using Microarray Suite 4.0 software (Affymetrix).

Reverse transcriptase polymerase chain reaction (RT-PCR): mRNA (poly-A positive) was reversed transcribed into cDNA following a standard protocol. Specific primers for selected gene sequences were designed. PCR and gel were run under standard conditions. To ensure that DNA product was generated at the exponential portion of the product curve, the following cycle numbers were used: 28 cycles for ACE; 32 cycles for Rap2 interacting protein and TNF-α; and 34 cycles for lipocalin 2, amphiregulin, leucine-rich-α2-glycoprotein, and angiotensin II receptors. Gel bands were analyzed by DNA sequencing to ensure the correct product. Kodak EDAS System (Rochester, N.Y.) was used for imaging and quantification. Results were expressed as the ratio of the investigated mRNA over the β-actin mRNA expression.

Real time PCR: Real-time PCR was run to better quantify actual changes in ACE expression. cDNA was also used for real time PCR. The same primers for ACE and β-actin were used as for conventional PCR, SYBR Green I was utilized for fluorescence. PCR was run with the following steps: 94° C. for 15 seconds, 66° C. for 15 seconds, and 72° C. for 25 seconds. A cDNA standard curve was created with defined concentrations of cDNA. This allowed for an extrapolation of the numbers of copies using the following formula: cDNA copies/ml=DNA concentration (mg/ml)×(10⁶ pg/mg)×(pmol/660 pg)×(1/sequence size [bp])×(10¹² mol/10³ ml)×6.023×10²³. Ct- values of ACE and β-actin were converted to numbers of cDNA copies, and results expressed as the ratio of ACE over β-actin expression.

Immunoblot analysis: Protein was extracted from isolated IEL and immunoblots performed by using a Bio-Rad protein assay kit (Bio-Rad Laboratories, Hercules, Calif.), with standard methods. Purified anti-ACE (Clone 3G8) (1:1000, Chemicon International, Temecula, Calif.) was used for ACE detection. Expression for β-actin was determined in the same fashion by re-probing membranes with purified anti-mouse β-actin (1:12000, Sigma-Aldrich, St. Louis, Mo.). Results are expressed as the ratio of ACE over β-actin protein expression.

Statistical analysis: Microarray gene analysis was performed using ArrayAssist software (Iobion Corp.). Initial analysis incorporated raw chip data and compared all study groups matched by week of study. Statistics utilized a modification of the paired t-test comparison consisting of a Benjamini-Hochberg correction (RMA analysis). Data criteria was considered significant if fold-change (FC) between groups was >2 and P<0.05 (F-test), or FC>3 and 0.05>P>0.01, or FC>4 and P>0.05. Significantly elevated genes were then selected based on identified gene functions relating to either cell proliferation and/or apoptosis. Remaining data are expressed as mean±standard deviation. These results were analyzed using ANOVA with least significant difference for post-hoc testing; statistical significance was set at P<0.05.

### EXAMPLE 2

### Microarray Analysis

Significant differential changes were noted in 65 genes at 1 week, and 254 genes after 4 weeks, between SBS and Sham groups. Genes were analyzed for relevance to proliferation or apoptosis. Five genes were identified (Table 1): angiotensin converting enzyme (ACE), lipocalin 2, Rap2 interacting protein, amphiregulin, and leucine-rich-α2-glycoprotein. Expression was confirmed for each with RT PCR. Although all five of these genes may have effects on EC growth, ACE was selected for additional examination due to its profound action on alveolar EC apoptosis.

TABLE 1
IEL-genes with greatest alterations in growth-modifying factors; shown are RT- PCR results
expressed as the relative expression compared to β-actin (mean ± standard deviation, n = 6.

| | | | Rap2 | | Leucine-rich- |
|---|---|---|---|---|---|
| | | Angiotensin | interacting | | α2-glyco- |
| | | converting | protein | | protein |
| Gene | Lipocalin 2 | enzyme | Apoptosis↓ | Amphiregulin | Apoptosis↑ |
| Function | Apoptosis↑ | Apoptosis↑ | Proliferation↑ | Proliferationt↑ | Proliferation↓ |
| Sham 1w mean | 0.72 ± 0.65†† | 0.93 ± 0.10† | 0.27 ± 0.17 | 0.31 ± 0.16 | 0.57 ± 0.28 |
| SBS 1w mean | 0.62 ± 0.13 | 1.04 ± 0.07 | 0.18 ± 0.13** | 0.18 ± 0.07** | 0.54 ± 0.11 |
| Sham 4w mean | 0.25 ± 0.22* | 0.89 ± 0.06†† | 0.26 ± 0.08 | 0.25 ± 0.09 | 0.34 ± 0.24* |
| SBS 4w mean | 0.45 ± 0.27 | 1.01 ± 0.24 | 0.21 ± 0.16 | 0.25 ± 0.11 | 0.47 ± 0.21 |

†P < 0.05 Sham 1w vs. SBR 1w,
*P < 0.05 Sham 4w vs. SBR 4w,
††P < 0.05 Sham 1w vs. Sham 4w,
**P < 0.05 SBR 1w vs. SBR 4w

### EXAMPLE 3

### ACE Expression Increases with Formation of SBS

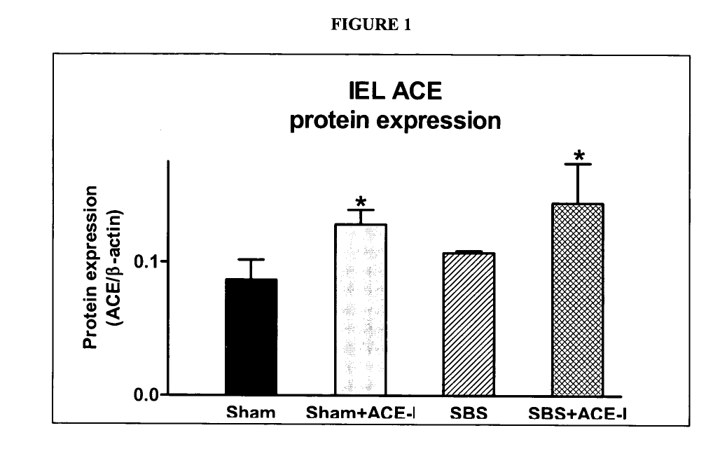

To confirm microarray findings, real-time PCR analysis was performed (Table 2). These results similarly showed a significantly (P<0.05) increased expression of IEL-derived ACE in the SBS 1 w mouse group. Western immunoblotting (Table 2, FIG. 1) showed that ACE protein expression increased by 17% in the SBS group, although the change was not significant.

TABLE 2
Summary of results of ACE-inhibitor (ACE-I) studies. Sham + ACE-I was used
as an additional control to assess the effects of ACE-I in the absence of a SBS.

| | | | EC | Villus | Crypt | |
|---|---|---|---|---|---|---|
| | IEL ACE | IEL ACE | Apoptosis^{b} | height | depth | IEL TNF-α |
| Group | mRNA^{a} | protein | (%) | (μm) | (μm) | mRNA |
| Sham | 0.22 ± 0.11 | 0.09 ± 0.02 | 3.6 ± 1.2 | 353 ± 40 | 77 ± 10 | 0.41 ± 0.13 |
| Sham + ACE-I | 0.66 ± 0.22† | 0.13 ± 0.01† | 1.9 ± 0.4† | 340 ± 20 | 87 ± 6 | 0.37 ± 0.07 |
| SBS | 0.49 ± 0.08* | 0.11 ± 0.01 | 4.3 ± 1.3 | 556 ± 66* | 113 ± 14* | 0.70 ± 0.19* |
| SBS + ACE-I | 0.63 ± 0.25 | 0.14 ± 0.03° | 1.8 ± 0.5° | 552 ± 56 | 145 ± 22° | 0.50 ± 0.07° |

^{a}Results of real time PCR (results are expressed as the ratio of ACE-cDNA copies over β-actin-cDNA copies).
^{b}Results of TUNEL analysis.
Abbreviations:
IEL, intraepithelial lymphocytes;
ACE, angiotensin converting enzyme;
EC, epithelial cells;
TNF-α, tumor necrosis factor alpha.
*P < 0.05 Sham vs. SBS,
°P < 0.05 SBS vs. SBS + ACE-I,
†P < 0.05 Sham vs. Sham + ACE-I

### EXAMPLE 4

### ACE Expression in IEL Subpopulation

IEL were sorted to determine if there was a differential expression of ACE in IEL sub-populations. IEL for this section were derived from adult, untreated mice. mRNA expression of ACE was detected in all sorted IEL sub-populations (Table 3). Although a greater expression of ACE was noted in the CD8α sub-population, no statistical difference was seen among groups.

TABLE 3
ACE mRNA expression in sorted IEL sub-populations. IEL were
derived from adult, untreated mice. Note the fairly uniform
expression of ACE in each subpopulation.
Abbreviations: TCR, T-cell receptor.

| IEL | ACE |
|---|---|
| Subpopulation | mRNA Expression |
| αβ-TCR | 0.63 ± 0.01 |
| γδ-TCR | 0.57 ± 0.15 |
| CD4 | 0.77 ± 0.13 |
| CD8α | 0.86 ± 0.13 |

### EXAMPLE 5

### Effect of ACE-I on EC Apoptosis, Histology and IEL ACE Expression

To further understand the relevance of ACE expression on EC apoptosis, ACE-inhibition was used. ACE-I (enalaprilat, 0.6 mg/kg/day i.p.) was given to both SBS (SBS+ACE-I), and sham operated (Sham+ACE-I) mice over a 7 day period, starting on the day after surgery. This latter group was studied as an additional control to assess the effects of ACE-I in the absence of a SBS. After 1 week, mice were killed, EC apoptotic rates assessed, and IEL mRNA and protein isolated.

Apoptosis:

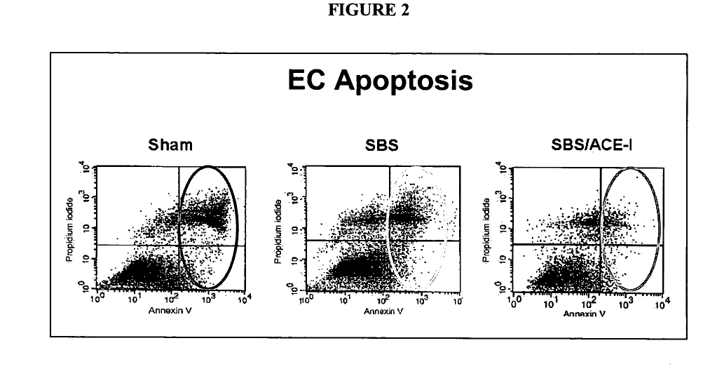

Annexin V staining: In the Sham and SBS groups EC apoptosis was significantly (P<0.01) increased (39.9±5.7% and 36.0±19.9%, respectively) compared to non-operated mice (14.4±5.1%)—a normal consequence of surgical resection. With ACE-I administration a significant decrease in apoptosis was noted in both Sham+ACE-I and SBS+ACE-I groups (16.9±8.8% and 13.1±4.5%, respectively) (FIG. 2). EC necrosis was also decreased in the enalaprilat treated mice. However, the differences were not significant between groups: Sham 11.6±7.1%, SBS 9.6±4.6, Sham+ACE-I 5.2±2.6%, SBS+ACE-I 5.2±2.6%.

TUNEL staining: As EC apoptosis rates tended to be high with Annexin V staining due to separation of EC tight junctions during the isolation process, a second, in situ method for measuring apoptosis was performed (Table 2). In the SBS+ACE-I-group the apoptotic rate declined by 58% compared to untreated SBS mice. In the Sham+ACE-I group EC apoptosis decreased by 48% compared to untreated Sham mice. Location of apoptotic cells was also altered with ACE-I administration. It was noted that apoptotic cells in SBS and Sham groups were distributed in both crypts and lower villi, whereas after administration with ACE-I apoptotic cells were confined to the crypts.

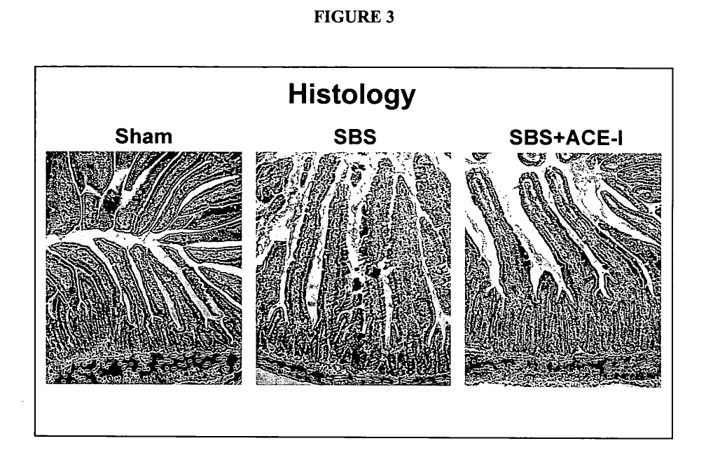

Histology: SBS group villus height and crypt depth were significantly increased compared to Sham mice. ACE-I administration to SBS mice resulted in a significant (P=0.02), additional increase in crypt depth, whereas villus height was unchanged compared to SBS mice (Table 2, FIG. 3). Sham+ACE-I mice showed no significant alterations in histological features.

### EXAMPLE 6

### Role of ACE Inhibitors in EC Apoptosis

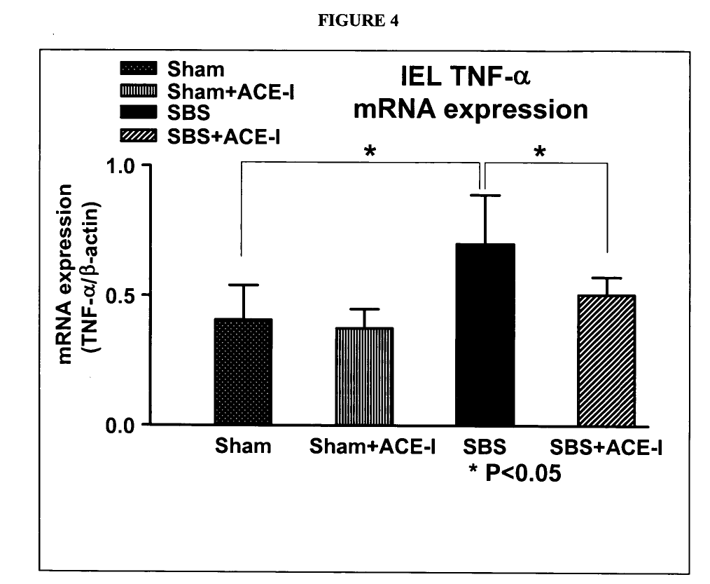

FIG. 4 shows that administration of ACE-I leads to a decreased expression of TNF-α.

### EXAMPLE 7

### Effects on Colitis

Male C57BL/6 specific-pathogen-free 2-month-old mice were kept in a 12-hour day/night environment maintained at 23° C. and a relative humidity of 40-60%. All mice drank 2.5% (w/v) DSS in water for 7 days followed by one day of plain water, while being fed normal mouse chow. Mice either received daily enalaprilat injection (0.015 mg/day, injection volume 0.5 ml, intraperitoneal) (n=6, “ACE-I”) or daily normal saline injection 0.5 ml (n=6, “Saline”). Body weight and hemoccult stool reaction were recorded. After the eight days of treatment, mice were euthanized with CO2, and colonic samples were immediately taken for histology and mucosal RNA extraction. Distal 1-cm colonic segments were excised, opened longitudinally, and fixed in 10% neutral buffered formalin. The remainder of the colon, including the cecum, was washed, cut into pieces, and mixed gently at 37° C. in EDTA/dithiothreitol buffer. The resulting mixture of epithelial cells and intraepithelial lymphocytes was then filtered through nylon wool and pelleted by centrifugation. Total RNA of the cell pellet was obtained using TRIZOL reagent and chloroform-isopropanol-ethanol extraction. Reverse transcription of RNA was done in the presence of RNAse inhibitor. RT product was then amplified with forward and reverse primers specific for ACE, TNF-α, and beta-actin, using real-time semiquantitative PCR. PCR results were analyzed by expressing ACE and TNF-α levels as a ratio to beta-actin levels, and were compared using a 2-tailed students' T test. Histologic grading of colitis was done by blinded observers using a standard scale for colitis severity. Histologic scores, as well as body weights and hemoccult results, were compared between groups using a 2-tailed homoscedastic T test.

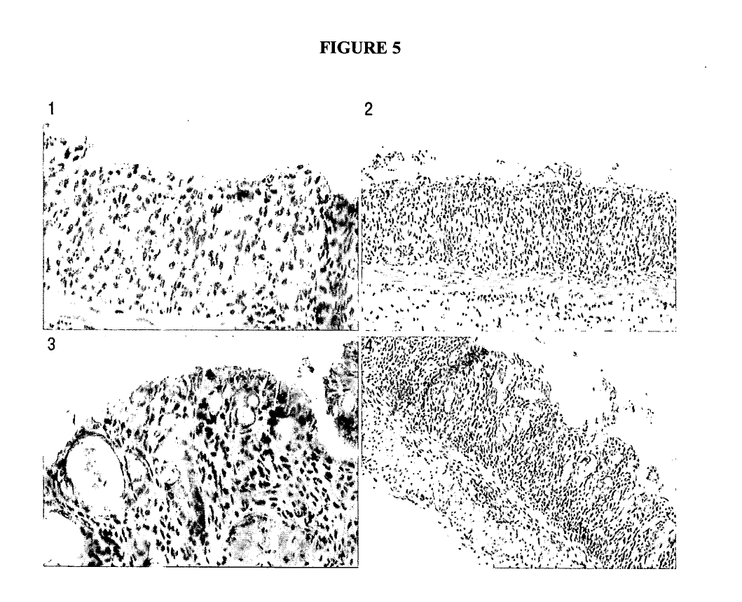

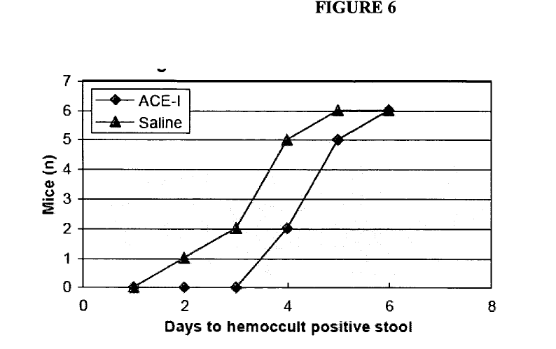

The clinical severity of colitis was improved by administration of ACE-I (FIG. 5 and Table 4) as shown by a significant prevention in loss of body weight, and a delay in the development of heme-positive stools. The time course of colitis is demonstrated in FIG. 6. ACE-I mice also showed significantly improved histologic colitis scores (12.6±1.6 vs. 14.8±1.3, p<0.01) (FIG. 5). These scores correspond to a uniformly severe ulcerative mucosal loss in Saline mice, but with partial sparing of colonic epithelium in ACE-I mice. Mucosal ACE levels were reduced in ACE-I mice (expressed in the table as a ratio to beta-actin levels).

TABLE 4
Results of outcomes in the two groups of mice: DSS treated with either
saline (DSS + Saline, Control group) or DSS treated with ACE-inhibitors
(DSS + ACE-I). Note the statistically significant improvement in
clinical outcomes of body weight loss, and onset of hemocult positive
stools in the ACE-I group. Additionally, note the significant improvement
in histologic grade in the ACE-I treated group.

| | Body Weight | Hemocult onset | Histologic | ACE levels |
|---|---|---|---|---|
| Groups | Loss (%) | (days) | Grade | (mRNA) |
| DSS + Saline | 23.7 ± 4.2 | 3.7 ± 1.0 | 14.7 ± 1.4 | 4.07 ± 4.71 |
| DSS + ACE-I | 16.6 ±+2.7 | 4.8 ± 0.8 | 11.8 ± 1.5 | 0.08 ± 0.13 |
| P level | <0.01 | <0.05 | <0.01 | <0.05, |
| | | | | one tailed |

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A method of treatment, comprising:
a) providing:
i) a subject having a symptom of inflammatory bowel disease or short bowel syndrome,
ii) a therapeutic composition comprising an angiotensin converting enzyme inhibitor, and;
b) administering said composition to said subject under conditions such that said symptom is reduced.

2. The method of claim 1, wherein said subject is a mammal.

3. The method of claim 2, wherein said mammal is a human.

4. The method of claim 1, wherein the route of administration of said composition is selected from intravenously, orally, parenterally, topically, and rectally.

5. The method of claim 1, wherein said composition further comprises a transdermal patch.

6. The method of claim 1, wherein said symptom of inflammatory bowel disease is associated with diseases selected from the group consisting of Crohn's disease, irritable bowel syndrome, celiac disease, ulcerative colitis, stomach ulcers, diverticulitis, pouchitis, proctitis or chronic diarrhea.

7. The method of claim 1, wherein said angiotensin converting enzyme inhibitor is selected from the group consisting of alacepril, benazepril (Lotensin), captopril (Capoten), cilazapril, ceranapril, delapril, enalapril (Vasotec), enalaprilat, fosinopril (Monopril), fosinoprilat, imidapril, lisinopril (Prinivil, Zestril), moexipril (Univasc), perindopril (Aceon), perindoprilat, quinapril (Accupril), quinaprilat, ramipril (Altace), saralasin acetate, spirapril, temocapril, trandolapril (Mavik), Bioproject BP1.137, Chiesi CHF 1514, Fisons FPL-66564, idrapril, Marion Merrell Dow MDL-100240, and Servier S-5590.

8. A method of treatment comprising:
a) providing:
i) a subject at risk for inflammatory bowel disease or short bowel syndrome, and
ii) a composition comprising angiotensin converting enzyme inhibitors, and;
b) administering to said subject a therapeutically effective amount of said composition so as to prevent said subject from experiencing symptoms of inflammatory bowel disease.

9. The method of claim 8, wherein said administration of said therapeutic composition delays the onset of symptoms of inflammatory bowel disease.

10. The method of claim 8, wherein said subject at risk for inflammatory bowel disease is a human.

11. The method of claim 10, wherein said human is selected from a child or adult, a person living in the United States, a person living in England, a person living in Northern Europe, a person of Jewish descent, a person living in a developing nation, a person with family members who suffer from inflammatory bowel disease or a person determined to carry an inflammatory bowel disease risk gene.

12. The method of claim 8, wherein the route of administration of said composition is selected from intravenously, orally, parenterally, topically, and rectally.

13. The method of claim 8, wherein said composition further comprises a transdermal patch.

14. The method of claim 8, wherein said subject at risk for inflammatory bowel disease is at risk for developing Crohn's disease, irritable bowel syndrome, celiac disease, ulcerative colitis, stomach ulcers, diverticulitis, pouchitis, proctitis or chronic diarrhea.

15. The method of claim 8, wherein said angiotensin converting enzyme inhibitor is selected from the group consisting of alacepril, benazepril (Lotensin), captopril (Capoten), cilazapril, ceranapril, delapril, enalapril (Vasotec), enalaprilat, fosinopril (Monopril), fosinoprilat, imidapril, lisinopril (Prinivil, Zestril), moexipril (Univasc), perindopril (Aceon), perindoprilat, quinapril (Accupril), quinaprilat, ramipril (Altace), saralasin acetate, spirapril, temocapril, trandolapril (Mavik), Bioproject BP1.137, Chiesi CHF 1514, Fisons FPL-66564, idrapril, Marion Merrell Dow MDL-100240, and Servier S-5590.

16. A pharmaceutical preparation comprising: an ACE inhibitor and a second compound capable of reducing a symptom of inflammatory bowel disease or short bowel syndrome.

17. The pharmaceutical preparation of claim 16, further comprising instructions for using said preparation for the treatment of inflammatory bowel disease.
